(19) United States
(12) Patent Application Publication
(10) Pub. No.: US 2007/0036732 A1
(43) Pub. Date: Feb. 15, 2007

(54) Stable topiramate formulations

(76) Inventors: Reza Eivaskhani, FREIBURG I.BR. (DE); Thomas Hunziker, Schaffhausen (CH); Rene Spycher, Benken (CH)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/538,674

(22) PCT Filed: Dec. 12, 2003
(86) PCT No.: PCT/EP03/14479

§ 371 Date: Nov. 1, 2006

(30) Foreign Application Priority Data
Dec. 13, 2002 (EP) 02080325.0

(51) Int. Cl. A61K 31/7008; A61K 9/68; A61K 9/24

(52) U.S. Cl. 424/48; 424/472; 514/23

(57) ABSTRACT

Bi- or multi-phasic tablets comprising an effective amount of a moisture sensitive active ingredient, which in particular is topiramate, wherein at least one of the phases comprises hygroscopic gum material, which preferably is xanthan gum, and wherein none of the phases contains both moisture sensitive active ingredient and hygroscopic gum material.

### FIELD OF THE INVENTION

This invention relates to bi- or multi-phasic tablets containing an active ingredient that is moisture sensitive, in particular topiramate, and at least one phase that comprises hygroscopic gum material and to processes for manufacturing such tablets.

### BACKGROUND OF THE INVENTION

Typical formulations that are used for oral administration of pharmaceutically active ingredients include liquid solutions, emulsions, or suspensions, as well as solid forms such as capsules or tablets (as used herein, the term “tablet” means any shaped and compressed solid dosage form, including caplets). Methods for preparing tablets are well known in the art, such that the drug agent contained therein is kept stable and active. Accordingly, tablet quality is measured against specifications such as appearance, hardness and drug agent availability as shown by dissolution rate and content uniformity.

Topiramate is a pharmaceutical active ingredient that is moisture sensitive and exposure to humidity causes its degradation. Topiramate belongs to a group of chlorosulfate and sulfamate esters of 2,3:4,5-bis-O-(1-methylethylidene)-β-D-fructopyranose, which compounds, their anticonvulsant activity in mammals and their utility in treating epilepsy are described in U.S. Pat. No. 4,513,006. More specifically, the compound 2,3:4,5-bis-O-(1-methylethylidene)-β-D-fructopyranose sulfamate, hereinafter referred to as “topiramate”, is presently commercially available as a tablet product in various strengths as adjunctive therapy for the treatment of adults with partial onset seizures. Topiramate can be prepared following the processes disclosed in U.S. Pat. Nos. 4,513,006 and 5,387,700 and, preferably, by the process described in Examples 1 to 3 of U.S. Pat. No. 5,387,700.

Exposure to moisture and heat,.though, causes the topiramate active agent in the solid dosage form to degrade. Topiramate in particular is very sensitive to water (humidity). Upon contact with humidity, topiramate degrades quickly and degradation accelerates because the degradation products have a catalytic effect on the degradation process itself. Degradation of topiramate tablets is readily detected by changes in physical appearance (discoloration of tablet color to brown or black) and by the formation of sulfate ions and organic degradation compounds, which can be readily detected by standard techniques known to those of ordinary skill in the art. Topiramate should therefore be well protected from moisture.

To further improve tablet quality and to prevent degradation of topiramate active ingredient, tablets are dried intensively to lower the amount of water in the tablets as much as possible to prevent the degradation of topiramate. Another reason for intensively drying the tablets is the so-called “greenhouse effect”. Any small amount of water that is present in topiramate tablets and/or that is locked into the cavities of a blister packaging negatively influences the stability of topiramate.

To maintain tablet quality, topiramate tablets have been packaged into both high-density polyethylene (HDPE) bottles containing a desiccant and blister packages containing a desiccant. Stability testing over time has demonstrated that the tablets in such packages have been preserved under various temperature, humidity and light conditions. Blister packages, however, offer advantages over the HDPE bottles used in the current marketed package and are, therefore, a preferred packaging format for topiramate tablets. Blister packages match the stability and marketing characteristics of HDPE bottles while being less expensive to package, lighter in weight and more conveniently stored; in addition, they offer rapid access, unit dose accountability and better physical protection for the product. Blister packages are especially advantageous for packaging moisture sensitive tablets such as topiramate because each tablet cavity then becomes a primary container in direct contact with the tablet, inherently enclosing a minimum of air and associated moisture.

Tablet stability in a blister package, therefore, becomes a function of the physical characteristics of the materials used in the composite blister package which affect permeability to moisture vapor and the ability to protect the enclosed product from light and humidity. Although other factors affect tablet stability, such as tablet moisture content and the packaging environment itself, manufacturers and packagers have focused on enhancing the stability and performance of blister packages by providing cavities for additional materials such as desiccants in addition to the tablet cavities.

WO-01/1304 describes pharmaceutical compositions comprising a combination of the analgesic tramadol and an anticonvulsant drug, in particular topiramate, useful to combat neuropathic pain. WO-99/44581 in turn discloses pharmaceutical compositions and their preparation comprising core particles containing topiramate wherein the particles have a taste mask coating. WO-02/102369 concerns the use of topiramate to protect retinal neurons.

WO-01/89445 describes a blister package for topiramate tablets which preserves stability of the active ingredient without a desiccant contained therein. This blister package comprises a pan sheet having preformed cavities containing pre-dried topiramate tablets and a cover sheet sealed to the pan sheet. Currently marketed topiramate tablets are packaged in the particular blister packages described in WO-01/89445. These packages are relatively expensive and the requirement for careful drying of the topiramate tablets prior to packing is cumbersome.

Hence there is a need for topiramate containing tablets that are stable in themselves and that do not need a pre-drying step or only need limited pre-drying. There is a further need for topiramate tablets that can be packed into standard blister packages. The bi- or multi-phasic tablets in accordance with the present invention are aimed at meeting these needs.

### SUMMARY OF THE INVENTION

The present invention is concerned with bi- or multi-phasic tablets comprising an effective amount of a moisture sensitive active ingredient, which in particular is topiramate, present in one or more of the phases, and wherein at least one of the phases comprises hygroscopic gum material and wherein none of the phases contains both moisture sensitive active ingredient and hygroscopic gum material.

In particular embodiments, the invention concerns a biphasic tablet having a phase that comprises an effective amount of a moisture sensitive active ingredient, in particular of topiramate, and another phase that comprises hygroscopic gum material.

In certain embodiments, the invention is concerned with bi- or multi-layer tablets comprising an effective amount of topiramate, wherein at least one of the layers comprises hygroscopic gum material and wherein none of the phases contains as well. topiramate and hygroscopic gum material.

Particular embodiments of the invention are bi-layer tablets having a layer that comprises an effective amount of topiramate and another layer that comprises hygroscopic gum material.

In any of the aspects or embodiments mentioned herein, the hygroscopic gum material in particular is alginate, gum Arabic or xanthan gum, the latter being preferred.

In a further aspect, there is provided a bi- or multi-phasic tablet, wherein at least one of the phases essentially consists of hygroscopic gum material and wherein none of the phases contains both topiramate and hygroscopic gum material.

In a specific aspect, there is provided a bi- or multi-layer tablet comprising an effective amount of topiramate, wherein at least one of the layers essentially consists of hygroscopic gum material and wherein none of the layers contains as well topiramate and hygroscopic gum material.

In a further aspect there is provided a process for manufacturing a bi- or multi-phasic tablet in accordance with the invention, said process comprising forming two or more pre-shaped phases and compressing the two or more pre-shaped phases in an appropriate compressing apparatus.

In a specific aspect there is provided a process for manufacturing a bi- or multi-layer tablet in accordance with the invention comprising compressing a suitable topiramate containing composition as to form a layer, laying a composition containing hygroscopic gum material on this topiramate containing layer, compressing the whole; and if desired laying further compositions of topiramate and/or further compositions containing hygroscopic gum material thereon and each time subjecting the whole to a compression; and if further desired coating the thus prepared dosage form.

In a further aspect there is provided a process for manufacturing a bi- or multi-layer tablet in accordance with the invention comprising compressing a composition containing hygroscopic gum material as to form a layer, laying a suitable topiramate containing composition on the hygroscopic gum material containing layer, and compressing the whole; and if desired laying further compositions containing topiramate and/or further compositions containing hygroscopic gum material thereon and each time subjecting the whole to a compression; and if further desired coating the thus prepared dosage form.

In a further aspect, there is provided a bi- or multi-phasic tablet, or a bi- or multi-layer tablet, in accordance with this invention, containing an effective amount of topiramate and having at least one phase or layer that contains from about 20% to about 100%, in particular from about 30% to about 90% or from about 50% to 80% of hygroscopic gum material.

In particular embodiments, the tablets according to the present invention are coated with an appropriate coating. The coating may be for taste masking or other purposes.

Furthermore, the invention concerns a method of treating a warm blooded animal suffering from epilepsy, said method comprising the administration of a bi- or multiphasic tablet containing an effective amount of topiramate, said tablet being as described herein.

### DETAILED DESCRIPTION OF THE INVENTION

Whenever used in this description and claims, any percentage is weight-by-weight.

The tablets of the invention contain a moisture sensitive active ingredient, which by preference is topiramate. The tablets of the invention further contain a hygroscopic gum material, which may be any gum material that is capable of taking up and retaining water. Of interest are those hygroscopic gums that are able to form a matrix and preferred gums are those which are mentioned above.

As used herein, ‘alginate’ refers to alginate or its salts, in particular to its alkali metal salts such as sodium or potassium salts. It is to be understood that although none of the phases contains both moisture sensitive active ingredient and hygroscopic gum material, any such phase containing moisture sensitive active ingredient may contain very small amounts of hygroscopic gum material and vice versa. As used in this context ‘very small amounts’ refers to amounts that do not affect the normal release of topiramate in that said release is slowed down, ‘very small amounts’ for example being less than 2%, in particular less than 1%, more in particular less than 0.5% (w/w relative to the total weight of the phase).

The tablets according to the invention contain at least two phases. As used herein the term ‘phase’ refers to a defined three dimensionally shaped section in a tablet dosage form that contains the same material and wherein each phase is separated from the other. The tablets of the invention may be bi-phasic, which is preferred, or multi-phasic, i.e. having 3,4, 5 or more phases. At least one phase should comprise hygroscopic gum material and at least another phase should comprise topiramate. In case of multiphasic tablets, more than one phase comprising hygroscopic gum material or more than one phase comprising topiramate can be present.

Examples of phases are layers, which are incorporated in bi- or multi-layer tablets. As used herein the term ‘layer’ in relation to tablets has its art-known meaning, i.e. a tridimensional section in a tablet usually of cylindrical shape with a relatively small thickness. Layers can have other shapes in case of tablets having a shape other than the usual round shaped tablet. Multi-layer tablets can be tablets with three, four, five, six or even more layers. Other examples are cylindrical, spherical or other tridimensionally shaped sections that can be present in tablets. This gives rise to different tablet forms such as the so-called ‘bull-eye’ tablets, or concentric tablets which have a central cylindrically shaped section surrounded with one or more further cylindrical layers (i.e. a ring-like combination), or ‘coated’ tablets wherein the coating in fact is a layer completely surrounding a tablet nucleus. Preference is given to bi- or multi-layer tablets.

Preferably a phase comprising hygroscopic gum material is adjacent to a phase containing topiramate.

Particular embodiments are tablets that contain topiramate, which may be present in amounts from about 10 mg to 500 mg topiramate per unit, preferably from about 25 mg to about 200 mg of topiramate per unit, e.g. tablets having 25, 50, 100 or 200 mg per unit.

In a particular aspect, the tablets of the invention contain an effective amount of topiramate, wherein the tablets have at least one phase, which may be a layer, that contains from about 20% to about 100%, in particular from about 30% to about 90% or from about 50% to 80% of hygroscopic gum material.

Each of the phases or of the layers in the tablets of the invention may additionally contain further ingredients such as starches, kaolin, lubricants, binders and the like.

Preferred additional carriers are lubricants, e.g. magnesium stearate, flow enhancers or fillers, e.g. silica (silicon dioxide), fillers such as sugars, in particular lactose, titanium dioxide and the like. Lactose may be added to improve compressibility of the blend. Magnesium stearate can be added to avoid tablet sticking on the lower or upper punch during the compression. The concentration of magnesium stearate in the tablets preferably is in the range of from about 0.5 to about 1.0% (w/w relative to the total weight of the tablet). The concentration of lactose in the tablets preferably is in the range of from about 5% to about 80%, preferably from about 10% to about 65%, more preferably from about 20% to about 50% (w/w relative to the total weight of the tablet).

In preferred embodiments, the hygroscopic gum material containing phase or layer contains as other ingredients one or more of lactose, magnesium stearate, silicon dioxide, and/or the topiramate phase or layer contains as further ingredients one or more of lactose and magnesium stearate.

The tablets of the invention can be prepared by mixing the active ingredient, which preferably is topiramate, with suitable carrier ingredients and compressing the mixture to a phase of desired shape, e.g. to a tablet layer. Different strengths of compression can be applied, e.g. complete or partial compression. The active ingredient containing mixture can be granulated and subsequently compressed or suitable mixtures containing active ingredient can be employed for direct compression, e.g. starting from suitable powdery mixtures. Subsequently the hygroscopic gum material is contacted with the compressed phase containing active ingredient forming another phase and the whole is compressed. In case of bi-layer tablets the hygroscopic gum material is laid onto the active ingredient layer so as to form another layer and the whole is compressed to a bi-layer tablet. Multi-layer tablets can be prepared by adding further layers of active ingredient and/or hygroscopic gum containing mixtures.

In a particular aspect the invention concerns a process for manufacturing a tablet as described herein, comprising direct compression of a mixture of an effective amount of topiramate with suitable ingredients as to form a layer and laying hygroscopic gum material on this layer and compressing the whole. In case of direct compression the other ingredients preferably are suitable fillers and suitable lubricants. The mixtures for direct compression preferably contain a lubricant, in particular magnesium stearate. They may additionally contain a filler, in particular a sugar such as lactose. They may furthermore contain a flow enhancer such as colloidal silica (silicon dioxide). In the mixtures for direct compression the lubricant preferably is present in concentrations in the range of about 0.75% to about 1.0%. The filler is present in concentrations from about 5% to about 80%, preferably from about 10% to about 65%, more preferably from about 20% to about 50%. The flow enhancer is present in concentrations from about 0.4% to about 0.6%, preferably about 0.45% to about 0.50%. All percentages herein are w/w relative to the total weight of the tablet.

Preferred embodiments of the invention are coated tablets, in particular film-coated tablets. Coated tablets are easier to swallow than uncoated tablet cores, are usually easier to distinguish from other tablets—in particular when the film-coat contains a dye or a pigment—, and may furthermore have improved stability (shelf-life). Coating can be done for taste masking purposes because of the bitter taste of topiramate. Coatings are applied using conventional methods using art known materials usually applied for this purpose.

Particularly attractive coating products are based on suitable film-forming polymers such as hydroxypropylmethylcellulose (HPMC) or polyvinylalcohol (PVA). Preferably, a plasticizer is added. Examples of suitable plasticizers are polyethylene glycol or derivatives thereof such as polyethoxylated alkylglycerides, e.g. polyethoxylated stearyl monoglyceride, in particular the material sold under the trade name Macrogol™ Further ingredients may be added to the coating such as fillers, dyes or pigments, flavors, sweeteners and the like components. Examples of such further ingredients are lactose, titanium dioxide, starch and the like. Particularly suited as coating materials are the Opadry™ materials, which mainly contain the before mentioned materials and further ingredients such as plasticizers, e.g. polyethylene glycol.

The tablets in accordance to the invention can be packed in standard blister packages without pre-drying, or when appropriate, with limited pre-drying. This makes the present tablets easier to handle and to package. The tablets of this present invention are easy to produce and are cost-effective because more simple and cheaper packaging techniques can be used and also the cumbersome drying step of the tablets prior to packaging can be avoided or limited.

Moreover the presence of a phase containing hygroscopic gum material has a positive effect on the stability of topiramate. Without being bound by theory, this can be explained by the fact that the hygroscopic gum material absorbs the water away from the topiramate containing phases or layers in the tablet.

### EXAMPLES

Formulation Example 1

Hygroscopic Gum Layer:

| Active and Excipients | mg/Tablet |
|---|---|
| Xanthan Gum | 130.00 |
| Lactose | 165.65 |
| Magnesium Stearate | 3.00 |
| Silicon Dioxide | 1.35 |
| Total | 300.00 |

Topiramate (Moisture Sensitive) Layer:

| Active and Excipients | mg/Tablet |
|---|---|
| Topiramate | 16.00 |
| Lactose Mohohydrate | 19.744 |
| Pregelatinized Starch | 4.096 |
| Microcrystalline Cellulose | 8.8 |
| Sodium Starch Glycolate | 2.56 |
| Magnesium Stearate | 0.256 |
| Total | 51.456 |

The ingredients listed in the table in the section ‘Topiramate layer’ are mixed and the mixture is compressed directly in a suitable tabletting machine as to form the topiramate layer. A mixture of the ingredients listed in the section ‘Hygroscopic gum layer’ is mixed and this mixture is layed on the topiramate layer and the whole is compressed a second time yielding a bi-layer tablet with a topiramate and a xanthan gum layer.

### Formulation Example 2

Hygroscopic Gum Material Layer:

| Active and Excipients | mg/Tablet |
|---|---|
| Xanthan Gum | 400.00 |
| Lactose | 43.25 |
| Magnesium Stearate | 4.50 |
| Silicon Dioxide | 2.25 |
| Total | 450.00 |

Topiramate (Moisture Sensitive) Layer:

| Active and Excipients | mg/Tablet |
|---|---|
| Topiramate | 128.00 |
| Lactose Monohydrate | 157.952 |
| Pregelatinized Starch | 32.768 |
| Microcrystalline Cellulose | 70.4 |
| Sodium Starch Glycolate | 20.48 |
| Magnesium Stearate | 2.048 |
| Total | 411.648 |

A bi-layer tablet is prepared similarly as outlined in example 1

1. A bi- or multi-phasic tablet comprising an effective amount of a moisture sensitive active ingredient, present in one or more of the phases, and wherein at least one of the phases comprises hygroscopic gum material and wherein none of the phases contains both moisture sensitive active ingredient and hygroscopic gum material.

2. A bi- or multiphase tablet according to claim 1 wherein the active ingredient is topiramate.

3. A bi-phasic tablet according to claim 2, having one phase that comprises an effective amount of a moisture sensitive active ingredient and another phase that comprises hygroscopic gum material.

4. A bi- or multi-phasic tablet according to claim 1, which are bi- or multi-layer tablets comprising an effective amount of topiramate, wherein at least one of the layers comprises hygroscopic gum material and wherein none of the phases contains as well topiramate and hygroscopic gum material.

5. A bi- or multi-phasic tablet according to claim 4, which is a bi-layer tablet, having a layer that comprises an effective amount of topiramate and another layer that comprises hygroscopic gum material.

6. A bi- or multi-phasic tablet according to claim 2, wherein the hygroscopic gum material is alginate, gum Arabic or xanthan gum.

7. A bi- or multi-phasic tablet according to claim 2, wherein the hygroscopic gum material is xanthan gum.

8. A bi- or multiphase tablet according to claim 2, containing at least one phase or layer that contains from about 20% to about 100%, in particular from about 30% to about 90% or from about 50% to 80% of hygroscopic gum material.

9. A bi- or multiphase tablet according to any of claim 2, wherein the tablet is coated with a suitable coating.

10. A bi- or multiphase tablet according to any of claim 9, wherein the tablet is coated with a HPMC or PVA.

11. A process for manufacturing a bi- or multi-phasic tablet as claimed in claim 1, said process comprising forming two or more pre-shaped phases and compressing the two or more pre-shaped phases in an appropriate compressing apparatus.

12. A process according to claim 11, comprising compressing a suitable topiramate containing composition as to form a layer, laying a composition containing hygroscopic gum material on this topiramate containing layer, compressing the whole; and if desired laying further compositions of topiramate and/or further compositions containing hygroscopic gum material thereon and each time subjecting the whole to a compression; and if further desired coating the thus prepared dosage form.

13. A process according to claim 11, comprising compressing a composition containing hygroscopic gum material as to form a layer, laying a suitable topiramate containing composition on the hygroscopic gum material containing layer, and compressing the whole; and if desired laying further compositions containing topiramate and/or further compositions containing hygroscopic gum material thereon and each time subjecting the whole to a compression; and if further desired coating the thus prepared dosage form.
